(19) United States
(12) Patent Application Publication
(10) Pub. No.: US 2007/0036864 A1
(43) Pub. Date: Feb. 15, 2007

(54) Hygeine product for mouth

(76) Inventors: Knut Yngve Börsheim, Trondheim (NO); Simon Ballance, Trondheim (NO); Bjorn Erik Christensen, Trondheim (NO); Olav Smidsrod, Trondheim (NO)

Correspondence Address: WENDEROTH, LIND & PONACK, L.L.P., 2033 K STREET N. W., SUITE 800, WASHINGTON, DC, 20006-1021, US

(21) Appl. No.: 10/577,306

(22) PCT Filed: Oct. 27, 2004
(86) PCT No.: PCT/NO04/00326

§ 371 Date: Aug. 30, 2006

(30) Foreign Application Priority Data
Oct. 30, 2003 (NO) 20034847

(51) Int. Cl. A61K 9/14

(52) U.S. Cl. 424/486

(57) ABSTRACT

An oral hygiene product, such as tooth paste, is described. A methos for controlling undesirable bacterial flora in the oral cavity, as well as the use of the plant Sphagnum, are also described.

The present invention relates to an oral hygiene product and a method for controlling undesirable bacterial flora in the oral cavity. The use of the plant Sphagnum in the production of tooth paste with antibacterial effect is also described.

The plant Sphagnum is known to possess antibacterial properties.

A hygienic tampon where Sphagnum is used for bactericidal and absorbing effect is described in A. P. Podterob and E. V. Zubets. A History of Medicinal Use of Plants of Genus Sphagnum. Medical Plants, Pharmaceutical Chemistry Journal, Vol. 36, No. 4, 2002.

Further, patent application RU 94002278/14 describes a tampon for use in dental surgery and in which Sphagnum is used as absorption material.

There is, however, no description of the use of the plant Sphagnum, or is parts thereof, in oral hygiene products such as tooth paste.

A tooth paste is a complex product which fulfils a series of different functions. The tooth paste should first and foremost clean the teeth when these are being brushed, as well as dissolve unwanted plaque in a gentle and hygienically safe way. Tooth paste should not contain antibacterial agents that are classified as antibiotics, but should nevertheless contain components that facilitate the removal of excess bacteria from teeth and oral cavity. Elements from the plant Sphagnum bind with organic particles in a mild and gentle way. This is conducive to tooth paste, containing elements of the plant Sphagnum, intercepting bacteria and remnants of food, which will be removed when the mouth is rinsed. Elements in the plant Sphagnum are not poisonous, and users who swallow the tooth paste by mistake will therefore not experience any problems because of the ingredients. Another advantage is that the elements of the plant Sphagnum are flavourless.

The present invention thus relates to the use of the plant Sphagnum, or extract thereof, together with conventional fillers in the production of tooth paste having antibacterial effect. According to the invention the plant Sphagnum, or extracts of it, can thus be used in tooth paste. It is also possible, in accordance with the invention, to use the plant Sphagnum, or an extract of it, in a composition for reducing the amount of bacterial flora on the teeth and in the oral cavity.

The present invention further relates to a mouth hygiene product, characterised in that it contains the plant Sphagnum, or extract thereof, together with conventional fillers.

The present invention also describes a method for controlling undesirable bacterial flora on the teeth and in the oral cavity by means of an oral hygiene product, preferably a tooth paste containing the plant Sphagnum, or extract thereof.

Oral hygiene products such as tooth paste contain a series of functional components and by adding the plant Sphagnum or parts thereof it is possible to increase the antibacterial effect of the product. The amount of bacterial flora on the teeth in the oral cavity will be reduced as a result of the antibacterial effect of the tooth paste of the invention.

The plant Sphagnum contains the substance Sphagnum holocellulose which is covalent bonded to cellulose and hemicellulose in the cell wall (Innovative Food Science and Emerging Technologies 2, 63-74; Børsheim et al. 2001 a; Carbohydrate Polymers 15, 123-142, Painter, 1991b). Sphagnum holocellulose has a structure which is very similar to the structure of typical complex pectins in many higher plants. It is a high molecular polysaccharide of about 40 kD. About 25% of Sphagnum holocellulose consists of a ketouronic acid where reactive carbonyl groups are found. In addition, Sphagnum contains holocellulose D-galacturonic acid and neutral sugar compounds. The reactive sugar, 5-keto-D-mannuronic acid (5KMA), bonds with the primary amines which are found on the surface of bacteria (Carbohydrate Polymers 36, 335-347, Painter, 1998). 5KMA exists as two isomeric ring forms in equilibrium. These are pyranose and furanose. The furanose form contains free carbonyl groups.

[image]

The carbonyl group in 5KMA condenses with ammonium or a primary amine and forms a Schiff's base (FIG. 1-2).

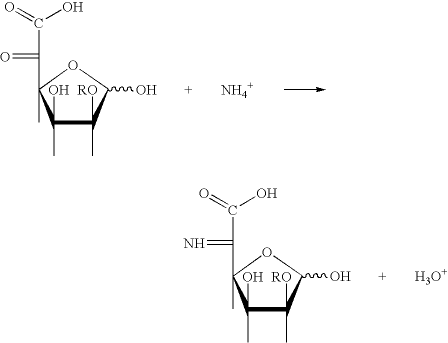

### EXAMPLES

Extraction of Sphagnum holocellulose

Sphagnum plant material was dried over night in an air flow at 60° C. and the leaves where then picket off. 50 g leaves were washed with 2 l boiling acetone at 57° C. for 3 minutes, and then isolated by filtration through a nylon filter with a pore size of 60 μm. This was repeated at least another three times, until the filtrate was almost colourless. Then the leaf remains were extracted once more with dry is methanol and air dried at 25° C.

20 g of this product was stirred mechanically in 3 l of water at 75° C. 30 ml glacial acetic acid was then added, followed by 30 g sodium chlorite added in portions over the course of 1 hour. After 3 hours these additions were repeated and after another 3 hours the mixture was cooled and filtrated. Pure white holocellulose was washed with water, then with glacial 0.02 M hydrochloric acid and then with distilled water until the wash water was neutral. The holocellulose was then washed with acetone and air dried in vent at room temperature. (Børsheim, K.Y. et al., Innovative Food Science and Emerging Technologies 2:63-74, 2001.)

Immobilisation of Whole Bacteria Cells

Sphagnum cellulose reacts with proteins (Chemistry and Industry, pp. 421-423, Painter, 1991 a). On the surface of bacteria there are numerous protein components and in this experiment immobilisation of bacteria cells is demonstrated.

Parts of Sphagnum palustre L. (Sphagnaceae) holocellulose were portioned in bags sewn of plankton cloth with a pore size of 20 μm. The bags were suspended separately in 0.9% NaCl solution in Erlenmeyer flasks with screw caps and autoclaved. The flasks were inoculated with varying parts of floating cultures of bacteria cells in their stationary growth phase, and transferred to a mechanic stirrer at 20° C. At different time intervals samples of the cell suspensions were taken out to determine bacterial density. It was shown that the density of the bacteria cells had been reduced to constant values after 20 hours. The results achieved with unmodified homocellulose are shown in Table 1. The controls were pure wood cellulose and Sphagnum holocellulose which had been treated with aqueous sodium boron hydride to reduce its carbonyl groups (Innovative Food Science Emerging Technologies, 2, 63-74, Børsheim et al., 2001 a). No significant immobilisation of bacteria cells was shown here. Unbleached moss and turf gave results that are comparable to those in Table 1 (Proceedings of the International Symposium or Microbiol. Ecology, Amsterdam, Aug. 21-26, 2001. Børsheim et al., 2001 b).

TABLE 1
Immobilisation of whole bacteria cells on Sphagnum holocellulose

| Culture | % remaining in suspension |
|---|---|
| Pseudomonas sp. | <0.4 |
| Escherichia coli | <0.25 |
| Bacillus sp. | <10 |
| Micrococcus sp. | <0.1 |

Table 1 shows that all the bacteria that were tested were immobilised by Sphagnum holocellulose. Controls with boron hydride reduced homocellulose showed no immobilisation. It should be noted that the bacteria that were tested were very different with regard to surface properties. Pseudomonas and Escherichia coli are gram negative, while Bacillus and Micrococcus are gram positive. It is therefore natural to assume that the plant Sphagnum or parts thereof possess immobilising properties with regard to most bacteria.

1 to 6. (canceled)

7. An oral hygiene product which contains the plant Sphagnum, or extract thereof, together with conventional fillers.

8. The oral hygiene product of claim 7 which is a tooth paste.

9. A method for controlling undesirable bacterial flora on teeth and in the oral cavity by means of an oral hygiene product containing the plant Sphagnum, or extract thereof.

10. The method of claim 9 wherein the product is a toothpaste.

11. A method for the production of tooth paste having antibacterial effect, which comprises mixing the plant Sphagnum, or extract thereof, together with conventional fillers.
